(19) United States
(12) Patent Application Publication
(10) Pub. No.: US 2005/0176793 A1
(43) Pub. Date: Aug. 11, 2005

(54) Amorphous form of 2-n-butyl-3-((2-(1h-tetrazol-5-yl)([1,1'-biphenyl)-4-yl)methyl)-1, 3-diazaspiro(4,4')non-1-en-4-one

(76) Inventors: Reguri Buchi Reddy, Hyderabad (IN); Sunkari Sudhakar, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 10/498,197

(22) PCT Filed: Dec. 6, 2002
(86) PCT No.: PCT/US02/39215

(30) Foreign Application Priority Data
Dec. 10, 2001 (IN) 992/MAS/2001

(51) Int. Cl. A61K 31/4188; C07D 43/02

(52) U.S. Cl. 514/381; 548/253

(57) ABSTRACT

The present invention relates to a novel amorphous form of 2-n-butyl-3-[[2′-(1H-tetrazol-5-yl)[1,1′-biphenyl]-4-yl]methyl]-1,3-diazaspiro[4.4]non-1-en-4-one and to a process for preparation thereof. Irbesartan (2-n-Butyl-3-[[2′-(1H-tetrazol-5-yl)[1,1′-biphenyl]-4-yl]methyl]-1,3-diazaspiro[4.4] non-1-en-4-one), represented by the following formula (I), is a non-peptide angiotensin—II antagonist. By inhibiting the action of angiotensin—II on its receptors, this compound prevents the increase in blood pressure produced by the hormone-receptor interactions and is hence used in the treatment of cardiovascular complaints such as hypertension and heart failure.

The present invention relates to a novel amorphous form of 2-n-butyl-3-[[2′-(1H-tetrazol-5-yl)[1,1′-biphenyl]-4-yl]methyl]-1,3-diazaspiro[4.4]non-1-en-4-one and to a process for preparation thereof.

Irbesartan (2-n-Butyl-3-[[2′-(1H-tetrazol-5-yl)[1,1′-biphenyl]-4-yl]methyl]-1,3-diazaspiro[4.4] non-1-en-4-one), represented by the following formula, 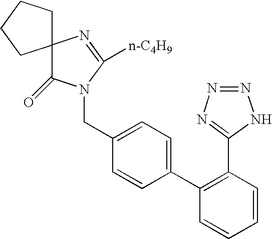
is a non-peptide angiotensin—II antagonist. By inhibiting the action of angiotensin—II on its receptors, this compound prevents the increase in blood pressure produced by the hormone-receptor interactions and is hence used in the treatment of cardiovascular complaints such as hypertension and heart failure.

U.S. Pat. No. 5,270,317 discloses certain N-substituted heterocyclic derivatives including 2-n-butyl-3-[[2′-(1H-tetrazol-5-yl)[1,1-biphenyl]-4-yl]methyl]-1,3-diazaspiro[4.4]non-1-en-4-one, its salts with acids or bases, methods of making and using them.

U.S. Pat. No. 5,629,331 discloses two polymorphic forms, Form-A and Form-B of 2-n-butyl-3-[[2′-(1H-tetrazol-5-yl)[1,1′-biphenyl]-4-yl]methyl]-1,3-diazaspiro[4.4]non-1-en-4-one, process for their preparation and use of the same for the treatment of hypertension.

WO patent application 99/67236 discloses a new crystalline habit of the Form-A of 2-n-butyl-3-[[2′-(1H-tetrazol-5-yl)[1,1′-biphenyl]-4-yl]methyl]-1,3-diazaspiro[4.4]non-1-en-4-one, process for its preparation and a composition containing it.

It has been disclosed in prior art that the amorphous forms of a number of drugs exhibit different dissolution characteristics and in some cases different bio-availability patterns when compared to crystalline forms (Konne T., Chem. Pharm. Bull., 38, 2003 (1990)). For some therapeutic indications one bioavailability pattern may be favoured over another.

Amorphous forms of a number of drugs have been disclosed to exhibit different dissolution characteristics and in some cases different bioavailability patterns when compared to crystalline forms. The present invention aims to provide a novel amorphous form of 2-n-butyl-3-[[2′-(1H-tetrazol-5-yl)[1,1′-biphenyl]-4-yl]methyl]-1,3-diazaspiro[4.4]non-1-en-4-one and a process for preparation thereof.

The present invention is directed to a novel amorphous form of Irbesartan. The present invention also provides a process for the preparation of novel amorphous form of Irbesartan from Irbesartan Form-A or Form-B.

The process for the preparation of novel amorphous form of Irbesartan from Form-A or Form B of Irbesartan, involves dissolution of Form-A or Form-B of Irbesartan in a mixture of solvents viz. C₁-C₃ haloalkane solvents and C₁-C₄ straight or branched chain alcohol solvents, at ambient temperature, followed by substantially-complete distillation of the solvent followed by drying to obtain the desired amorphous form of Irbesartan.

The process for preparation of novel amorphous form of Irbesartan from Form-B of Irbesartan, involves dissolution of Form-B of Irbesartan in a single solvent viz. C₁-C₃ haloalkane solvent at ambient temperature followed by substantially-complete distillation of the solvent then followed by drying to obtain the desired amorphous form of Irbesartan.

### BRIEF DESCRIPTION OF THE ACCOMPANYING DRAWINGS

FIG. 1. is X-ray powder diffractogram of novel amorphous form of Irbesartan.

FIG. 2. is the DSC thermogram of the novel amorphous form of Irbesartan.

FIG. 3. is the IR spectra of the novel amorphous form of Irbesartan.

### DETAILED DESCRIPTION OF THE INVENTION

The present invention provides a novel amorphous form of Irbesartan. The present invention also provides processes for preparation of novel amorphous form of Irbesartan.

The novel amorphous form of Irbesartan of the present invention is prepared by a process, which comprises:
ii. dissolving Form-A or Form-B of Irbesartan in a mixture of C₁-C₃ haloalkane solvent and C₁-C₄ straight or branched chain alcohol solvents, or a mixture thereof, at ambient temperature;
ii. substantially distilling off the solvent from the solution obtained in step i) and
iii. drying the product obtained in step ii) to obtain the desired amorphous form of Irbesartan.

Preferably, the distillation of the solvent is carried out under reduced pressure.

Preferably, the ratio of Form-A to a mixture of C₁-C₃ haloalkane solvent and C₁-C₄ straight or branched chain alcohol solvent, is 1:2-20 weight/volume, wherein the ratio of C₁-C₃ haloalkane solvent to C₁-C₄ straight or branched chain alcohol solvent, is 1-10:10-1 volume/volume. More preferably, the ratio of C₁-C₃ haloalkane solvent to C₁-C₄ straight or branched chain alcohol solvent 4-10:10-4 v/v.

The ratio of Form-B to a mixture of C₁-C₃ haloalkane solvent and C₁-C₄ straight or branched chain alcohol solvent is 1:5-25 weight/volume wherein the ratio of C₁-C₃ haloalkane solvent to C₁-C₄ straight or branched chain alcohol is 1-5:4-20 volume/volume, preferably 1-5:5-10v/v.

The C₁ to C₃ haloalkane solvent employed for the dissolution of Form-A of Irbesartan or Form-B of Irbesartan is selected from dichloromethane, 1,2-dichloroethane and chloroform, preferably dichloromethane. The C₁-C₄ straight or branched chain alcohol solvents employed in case of Form-A or Form B of Irbesartan in admixture with the C₁ to C₃ haloalkane solvent are selected from methanol, ethanol, n-propanol, iso-propanol, n-butanol, iso-butanol and tert.-butanol, preferably methanol.

In the case of Form A, preferably, the ratio of dichloromethane to methanol is 4-10:10-4 v/v and in the case of Form B the ratio is 1-5:5-10 v/v.

Form-A and Form-B of Irbesartan may be prepared by any process known in the art. Form A and Form B may be prepared by the process disclosed in U.S. Pat. No. 5,629,331 which comprises:
(a) treating the 2-n.butyl-3-[(2′-cyanobiphenyl-4-yl)methyl]-1,3-diazaspiro-[4.4]-non-1-en-4-one with an alkaline azide and triethylamine hydrochloride in an inert polar aprotic solvent and the recovering the thus obtained 2-n.butyl-3-[[2′-(tetrazol-5-yl)biphenyl-4-yl]methyl]-1,3-diazaspiro[4.4]non-1-en-4one in the form of one of its alkaline salts in aqueous solution;
(b) neutralizing the alkaline salt 2-n.butyl-3-[[2′-(tetrazol-5-yl)biphenyl-4-yl]methyl]-1,3-diazaspiro[4.4]non-1-en-4-one in aqueous medium until the pH is of from 4.7 to 5.3; and
(c) crystallizing the precipitated product:
   either in a solvent containing less than about 10% in volume of water to isolate the 2-n.butyl-3-[[2′-(tetrazol-5-yl)biphenyl-4-yl]methyl]-1,3-diazaspiro[4.4]non-1-en-4-one in its Form A;
   or in a water-miscible solvent containing more than about 10% in volume of water to isolate the 2-n.butyl-3-[[2′-(tetrazol-5-yl)biphenyl-4-yl]methyl]-1,3-diazaspiro[4.4]non-1-en-4-one in its Form B.

Form A of Irbesartan may also be prepared by the process as disclosed in our Indian Co-pending Application No. 809/MAS/2001 for preparation of Form-A of Irbesartan which comprises recrystallising the crude Irbesartan or Form-B of Irbesartan from ketone solvents selected from acetone, methyl ethyl ketone, dimethyl ketone or methyl isobutyl ketone, preferably methyl isobutyl ketone followed by optionally subjecting the resulting reaction solution to carbon treatment, followed by filtration. The filtrate so obtained is cooled to 0-5° C. under stirring and the resultant product is isolated by filtration to yield Irbesartan Form-A.

Form B may be prepared by the process disclosed in our Indian Co-pending Application No. 809/MAS/2001 for which Form-A of Irbesartan in a solution of inorganic base (viz. sodium hydroxide, potassium hydroxide, ammonium hydroxide, sodium carbonate, potassium carbonate) or organic base (viz. dimethylamine, triethylamine, tributylamine) followed by acidification using inorganic or organic acid (viz. hydrochloric acid, sulphuric acid, acetic acid) accompanied by stirring. The product that separates out is filtered and dried to yield Irbesartan Form-B.

The present invention also envisages a pharmaceutical composition comprising the novel amorphous of 2-n-butyl-3-[[2′-(1H-tetrazol-5-yl)[1,1′-biphenyl]-4-yl]methyl]-1,3-diazaspiro [4.4] non-1-en-4-one and a pharmaceutically acceptable carrier, diluent, excipient, additive, filler, lubricant, binder, stabilizer, solvent or solvate.

The pharmaceutical composition may be in a form normally employed, such as tablets, capsules, lozenges, powders, syrups, solutions, suspensions, ointments, dragees and the like, may contain flavourants, sweetners, etc. in suitable solid or liquid carriers or diluents, or in suitable sterile media to form injectable solutions or suspensions. Such compositions typically contain from 1 to 25%, preferably 1 to 15% by weight of active ingredient, the remainder of the composition being one or more of a pharmaceutically acceptable carrier, diluent, excipient, additive, filler, lubricant, binder, stabilizer, solvent or solvate.

The amorphous form of 2-n-butyl-3-[[2′-(1H-tetrazol-5-yl)[1,1′-biphenyl]-4-yl]methyl]-1,3-diazaspiro [4.4] non-1-en-4-one is administered to mammals, including man, via either oral, nasal, pulmonary, transdermal or parenteral, rectal, depot, subcutaneous, intravenous, intraurethral, intramuscular, intranasal, ophthalmic solution or an ointment. Administration by the oral route is preferred, being more convenient and avoiding the possible pain and irritation of injection. However, in circumstances where the patient cannot swallow the medication, or absorption following oral administration is impaired, as by disease or other abnormality, it is essential that he drug be administered parenterally. By either route, the dosage is in the range or about 0.01 to about 100 mg/kg body weight of the subject per day or preferably about 0.01 to about 100 mg/kg body weight of the subject per day or preferably about 0.01 to about 30 mg/kg body weight per day administered singly or as a divided dose. However, the optimum dosage for the individual subject being treated will be determined by the person responsible for treatment, generally smaller doses being administered initially and thereafter increments made to determine the most suitable dosage.

Suitable pharmaceutically acceptable carriers include solid fillers or diluents and sterile aqueous or organic solutions. The active ingredient will be present in such pharmaceutical compositions in the amounts sufficient to provide the desired dosage in the range as described above. Thus, for oral administration, the compound can be combined with a suitable solid or liquid carrier or diluent to form capsules, tablets, powders, syrups, solutions, suspensions and the like. The pharmaceutical compositions, may, if desired, contain additional components such a flavourants, sweeteners, excipients and the like. For parenteral administration, the compound can be combined with sterile aqueous or organic media to form injectable solutions or suspensions. For example, solutions in sesame or peanut oil, aqueous solutions of water-soluble pharmaceutically-acceptable acid addition salts or salts with base of the compounds. Aqueous solutions with the active ingredient dissolved in polyhydroxylated castor oil may also be used for injectable solutions. The injectable solutions prepared in this manner can then be administered intravenously, intraperitoneally, subcutaneously, or intramuscularly, with intramuscular administration being preferred in humans.

For nasal administration, the preparation may contain the compound of the present invention dissolved or suspended in a liquid carrier, in particular an aqueous carrier, for aerosol application. The carrier may contain additives such as solubilizing agents, such as propylene glycol, surfactants, absorption enhancers such as lecithin (phosphatidylcholine) or cyclodextrin or preservatives such as parabenes.

Tablets, dragees or capsules having talc and/or a carbohydrate carried binder or the like are particularly suitable for any oral application. Preferably, carriers for tablets, dragees or capsules include lactose, corn starch and/or potato starch. A syrup or elixir can be used in cases where a sweetened vehicle can be employed. An effective amount means that amount of a drug or pharmaceutical agent that will elicit the biological or medical response of a tissue, system, human or animal sought.

The following examples illustrate the invention but do not construe to limit the same.

### EXAMPLES

### Amorphous Irbesartan from Form-A of Irbesartan

### Example 1

To a suspension of Form-A of Irbesartan (5.0 g), in dichloromethane (50.0 ml) was added methanol (20.0 ml) at ambient temperature to get a clear solution. The solvents were then substantially distilled off under reduced pressure at a bath temperature of below 50° C. and the resultant product dried at 73°-80° C. to obtain the amorphous Irbesartan. (wt: 4.0 g).

### Example 2

To a suspension of Irbesartan Form-A (10.0 g) in chloroform (100.0 ml) was added methanol (40.0 ml) at an ambient temperature to obtain a clear solution. The solvents were then substantially distilled off under reduced pressure at a bath temperature of below 50° C. and the resultant product dried at 67°-72° C. to obtain the amorphous form of Irbesartan. (wt: 7.8 g).

### Amorphous Irbesartan From Irbesartan Form-B

### Example 3

Irbesartan Form-B (1.0 g,) was dissolved in a mixture of methanol (1.0 ml) and dichloromethane (5.0 ml) at ambient temperature to get a clear solution. The solvent was then substantially distilled off under reduced pressure at a bath temperature of below 50° C. and the resultant product dried at 67°-70° C. to get the amorphous Irbesartan. (wt: 0.5 g.).

### Example 4

Irbesartan Form-B (3.0 g,) was dissolved in a mixture of methanol (15 ml) and dichloromethane (15.0 ml) at ambient temperature to get a clear solution. The solvent was then substantially distilled off under reduced pressure at a bath temperature of below 50° C. and the resultant product dried at 67°-70° C. to get the amorphous Irbesartan. (wt: 2.1 g.).

### DETAILED DESCRIPTION OF THE ACCOMPANYING DRAWINGS

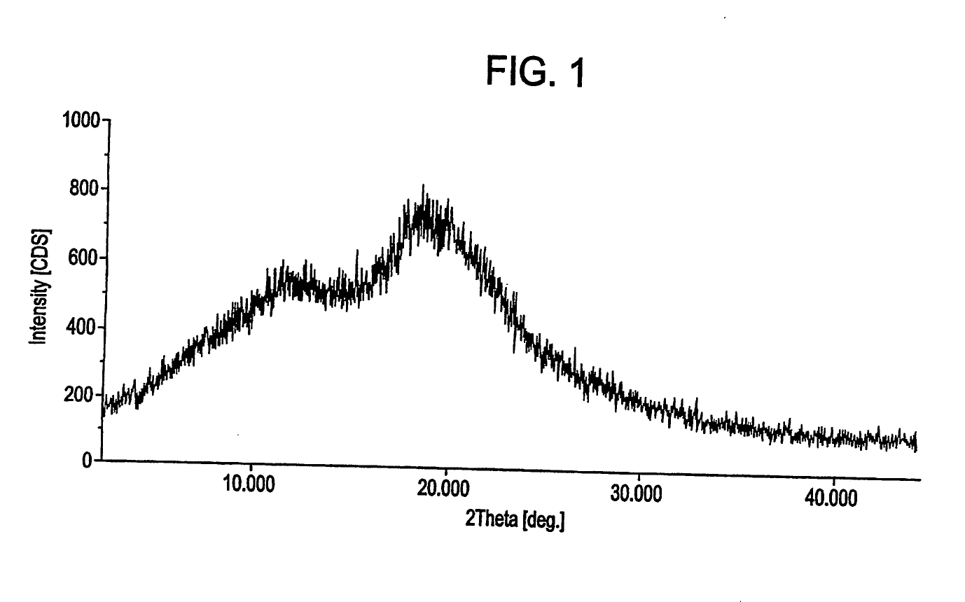

FIG. 1 is characteristic X-ray powder diffraction pattern of amorphous form of Irbesartan of this invention. Vertical axis: Intensity (CPS); Horizontal axis: 2 Theta (degrees). It shows a plain halo with no peaks, which is a characteristic of the amorphous nature of the product. Equipment: Rigaku DMax 2000, Radiation: Cu-K Alpha1/50KV/34 mA, Degrees Scanned: 3-45 deg.

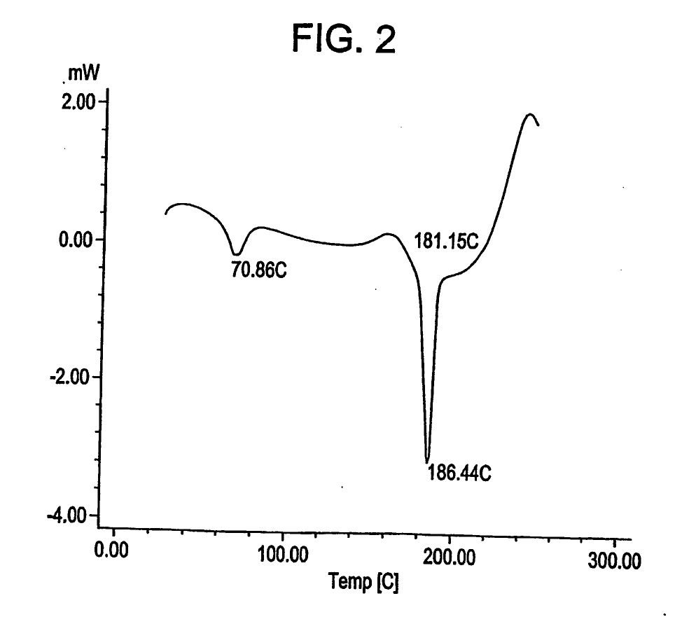

FIG. 2 is the DSC thermogram for the amorphous form of Irbesartan of this invention. The DSC thermogram shows a significant endo-endo pattern with peak temperatures at about 70.86° C. and 186.44° C. The sample was analyzed in a temperature range of 25-250° C. with a flow rate of 10° C./minute.

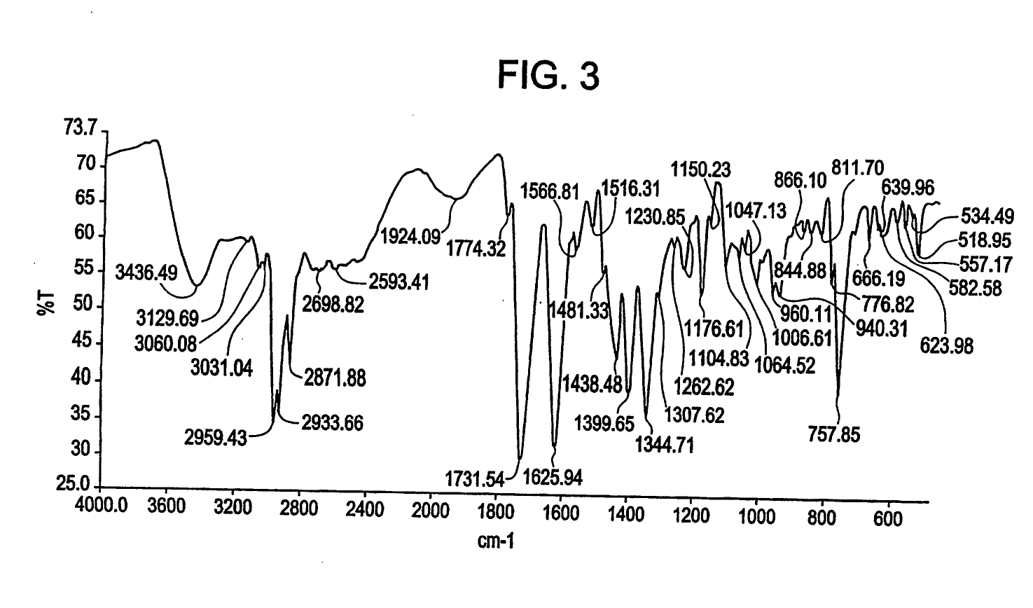

FIG. 3. Shows an IR spectra of the amorphous form of Irbesartan IR(cm⁻¹) of this invention.
Equipment: Perkin Elmer
Method: KBr Pellet
Range: 400-4000 cm−1 (Wavelength)

According to FIG. 3 the IR (cm−1) is as 3436.49, 3129.69, 3060.08, 3031.04, 2959.43 2933.66, 2871.88, 2698.82, 2593.41, 1924.09, 1774.32, 1731.54, 1625.94, 1566.81, 1516.31, 1481.33, 1438.48, 1399.65, 1344.71, 1307.62, 1262.62, 1230.85, 1176.61, 1150.23, 11-04.83,1064.52, 1047.13, 1006.61, 960.11, 940.31, 866.10, 844.88, 811.7, 776.82, 757.85, 666.19, 639.96, 623.98, 582.58, 557.17, 534.49, 518.95.

1. A novel amorphous form of 2-n-butyl-3-[[2′-(1H-tetrazol-5-yl)[1,1′-biphenyl]-4-yl]methyl]-1,3-diazaspiro [4.4] non-1-en-4-one.

2. The amorphous form according to claim 1, characterized by an X-ray powder diffraction pattern substantially as depicted in FIG. 1.

3. The amorphous form according to claim 1, characterized by DSC 70.86° C. (endotherm) and 186.44° C. (endotherm).

4. The amorphous form according to claim 1, characterized by the IR spectra of FIG. 3.

5. A process for preparation of amorphous form of 2-n-butyl-3-[[2′-(1H-tetrazol-5-yl)[1,1′-biphenyl]-4-yl]methyl]-1,3-diazaspiro[4.4]non-1-en-4-one which comprises:
i) dissolving Form-A or Form B of Irbesartan in a mixture of C₁-C₃ haloalkane solvent and C₁-C₄ straight or branched chain alcohol solvent, at ambient temperature;
ii) substantially distilling off the solvent from the solution obtained in step i) and
iii) drying the product obtained in step ii) to obtain the desired amorphous form of Irbesartan.

6. The process according to claim 5, wherein the ratio of Form-A to a mixture of C₁-C₃ haloalkane solvent and C₁-C₄ straight or branched chain alcohol solvent is 1:2-20 weight/volume.

7. The process according to claim 6, wherein the ratio of C₁-C₃ haloalkane solvent to C₁-C₄ straight or branched chain alcohol solvent, is 1-10:1-10 v/v.

8. The process according to claim 7, wherein the ratio of C₁-C₃ haloalkane solvent to C₁-C₄ straight or branched chain alcohol solvent 4-10:10-4 v/v.

9. The process according to claim 5, wherein the ratio of Form B to a mixture of C₁-C₃ haloalkane solvent and C₁-C₄ straight or branched chain alcohol is 1:5-25 weight/volume.

10. The process according to claim 9, wherein the ration of C₁-C₃ haloalkane solvent to C₁-C₄ straight or branched chain alcohol solvent, is 1-5:4-20 v/v.

11. The process according to claim 10, wherein the ratio of C₁-C₃ haloalkane solvent to C₁-C₄ straight or branched chain alcohol solvent 1-5:5-10 v/v

12. The process according to any one of claims 5 to 11, wherein the C₁-C₃ haloalkane solvent is selected from dichloromethane, 1,2-dichloroethane or chloroform.

13. The process according to any one of claims 5 to 11, wherein the C₁-C₄ straight or branched chain alcohol solvent is selected from methanol, ethanol, n-propanol, iso-propanol, n-butanol, iso-butanol or tertiary butanol.

14. The process according to any one of claims 5 to 12, wherein the C₁-C₃ haloalkane solvent is dichloromethane.

15. The process according to any one of claims 5 to 12, wherein the C₁-C₃ haloalkane solvent is chloroform.

16. The process according to any one of claims 5 to 12, wherein the C₁-C₄ straight or branched chain alcohol solvent is methanol.

17. The process according to any one of claims 5 to 8 or 12-16, wherein the ratio of dichloromethane to methanol is 4-10:10-4 v/v.

18. The process according to any one of claims 5 or 9-16, wherein the ratio of dichloromethane to methanol is 1-5:5-10 v/v.

19. The process according to any one of claims 5 to 18, wherein the distillation of step ii) is carried out reduced pressure.

20. A composition comprising an amorphous form of 2-n-butyl-3-[[2′-(1H-tetrazol-5-yl)[1,1′-biphenyl]-4-yl]methyl]-1,3-diazaspiro[4.4]non-1-en-4-one according to anyone of claims 1 to 4 and pharmaceutically acceptable carrier, diluent, excipient, additive, filler, lubricant, binder, stabilizer, solvent or solvate.

21. The composition according to claim 21, in the form of a tablet, capsule, lozenge, powder, syrup, solution, suspension, ointment, or dragee.

22. A composition according to claim 20 or 21, for the treatment of hypertension or heart failure.

23. (canceled)

24. (canceled)

25. A method for treating hypertension or heart failure comprising administering an effective amount of an amorphous form of 2-n-butyl-3-[[2′-(1H-tetrazol-5-yl)[1,1′-biphenyl]-4-yl]methyl]-1,3-diazaspiro[4.4]non-1-en-4-one according to any one of claims 1 to 4 and a pharmaceutically acceptable carrier, diluent, excipient, additive, filler, lubricant, binder, stabilizer, solvent or solvate to a patient in need thereof.
